(19) United States
(12) Patent Application Publication
(10) Pub. No.: US 2004/0161392 A1
(43) Pub. Date: Aug. 19, 2004

(54) Skin peeling composition and method

(75) Inventors: Isabelle Hansenne, Westfield, NJ (US); Hani Fares, Somerset, NJ (US); Marc Cornell, Jackson, NJ (US); Sidney P. Foltis, Nutley, NJ (US)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL S.A., Paris (FR)

(21) Appl. No.: 10/367,952

(22) Filed: Feb. 19, 2003

(51) Int. Cl. A61K 31/60; A61K 7/06

(52) U.S. Cl. 424/70.1; 514/159

(57) ABSTRACT

The present invention relates to methods of peeling skin using certain salicylic acid derivatives, to chemical skin peel compositions containing these certain salicylic acid derivatives in a carrier, preferably a dermatologically acceptable carrier, to methods of making these compositions, and methods of applying this certain compound and/or composition to skin to be peeled.

### BACKGROUND OF THE INVENTION

1. Field of the Invention

The present invention relates to methods of peeling skin using certain salicylic acid derivatives, to chemical skin peel compositions comprising these certain salicylic acid derivatives in a carrier, preferably a dermatologically acceptable carrier, to methods of making these compositions, and methods of applying this certain derivative and/or composition to skin to be peeled.

2. Discussion of the Background of the Invention

Conventional skin peeling procedures include mechanical removal, e.g., dermabrasion or CO2 laser, and chemical-induced skin removal. Chemical skin peeling techniques are currently very popular and are often categorized by the degree or amount of skin removal effected.

Chemical peels may be categorized as superficial, medium and deep chemical peels, depending on the depth of chemical wounding of the skin that occurs. Superficial chemical peels are those which remove or effect accelerated replacement or replenishment of the epidermis. Medium depth peels penetrate to the papillary dermis. Deep peels penetrate to the reticular dermis. WO 97/28786 reports that chemical peeling agents include α-hydroxy acids, e.g., glycolic acid or other “fruit acids” such as citric and lactic acids; trichloroacetic acid; phenol, resorcinol and Jessner's solution, an ethanol solution containing equal parts (about 14%) of resorcinol, salicylic acid and lactic acid (85%). However, a need exists for a chemical skin peeling agent and composition and related technique that provides exceptional results, preferably without or with less adverse side effects or drawbacks found with conventional chemical peels, agents and compositions.

### DETAILED DESCRIPTION OF THE PRESENT INVENTION

Unless specifically defined, all technical and scientific terms used herein have the same meaning as commonly understood by a skilled artisan in biochemistry, chemistry, cosmetology, dermatology and materials science.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting.

The term “accelerating the replacement or replenishment of epidermis” for the purposes of the present invention is intended to mean, for example, an increase or enhanced replacement or replenishment of epidermis in the presence of or as caused by the invention salicylic acid derivative and/or peeling method relative to replacement or replenishment of epidermis in the absence of the salicylic acid derivative. Such acceleration may be of any amount, for example any amount greater than 100% (e.g., 101, 103, 105%) including for example 1000% relative to the replacement or replenishment of epidermis in the absence of the salicylic acid derivative. 100% would denote no acceleration.

The present invention provides a method for chemically peeling the skin, comprising applying to skin to be peeled at least one salicylic compound (also referred to as a salicylic acid derivative) of formula (I):

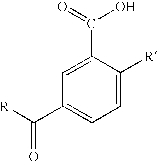

where

R is a linear, branched or cyclic saturated aliphatic group or an aliphatic unsaturated group containing one or a number of double bonds, which may or may not be conjugated, these groups containing from 2 to 22, preferably 3 to 11, carbon atoms and being able to be substituted for example by at least one substituent selected from (a) halogen atoms, (b) the trifluoromethyl group, (c) hydroxyl groups in the free form or esterified by an acid having from 1 to 6 carbon atoms or (d) a carboxyl functional group which is free or esterified by a lower alcohol having from 1 to 6 carbon atoms;

R′ is a hydroxyl group or an ester functional group of formula

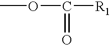

where R₁ is a linear or branched saturated or unsaturated aliphatic group having from 1 to 18 carbon atoms, and salts thereof.

In a preferred embodiment R is a linear, branched or cyclized saturated aliphatic chain containing from 3 to 11 carbon atoms, an unsaturated chain containing from 3 to 17 carbon atoms and containing one or more conjugated or unconjugated double bonds, the abovementioned chains being optionally substituted by one or more halogen atoms or by trifluoromethyl groups, by one or more hydroxyl groups in free form or esterified by an acid containing from 1 to 6 carbon atoms, or by a carboxyl group, free or esterified by a lower alcohol containing from 1 to 6 carbon atoms, these various groups being optionally simultaneously present in the said substituents; and

R′ is a hydroxyl group or an ester group of formula

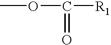

where R₁ is a saturated or unsaturated aliphatic group containing from 1 to 18 carbon atoms.

The present invention also provides a chemical skin peel composition comprising at least one of the above salicylic compounds and a carrier, preferably a dermatologically acceptable carrier, said composition being characterized in that it preferably has a concentration of salicylic compound of from 0.01 to about 35-36%, including all subranges and values therebetween, for example 0.5, 1, 3, 5, 7, 9, 11, 15, 18, 20, 22, 25, 27, 30, etc., %, etc., all percents being based on total weight of composition. Preferably the composition is one designed to be, and capable of being, rinsed off after application.

The present invention also provides methods of making the above-mentioned chemical skin peel compositions by mixing at least one salicylic acid derivative as described above with at least one carrier such as a dermatologically acceptable carrier, where “mixing” includes all orders of addition.

The present invention also provides chemical skin peel compositions that comprise at least one salicylic acid derivative as described above formulated so as to be acceptable to the consumer and, preferably, stable and/or clear.

Preferred salicylic acid derivatives useful herein include those described in U.S. Pat. No. 5,558,871, FR 2,581,542, U.S. Pat. No. 4,767,750, EP 378,936, U.S. Pat. No. 5,267,407, U.S. Pat. No. 5,667,789, U.S. Pat. No. 5,580,549, and EP-A-570,230, all incorporated herein by reference. Further, particularly preferred salicylic acid derivatives useful herein include 5-n-octanoyl salicylic acid (capryloyl salicylic acid), 5-n-decanoyl salicylic acid, 5-n-dodecanoyl salicylic acid, 5-n-heptyloxy salicylic acid and 4-n-heptyloxy salicylic acid. A highly preferred salicylic acid derivative is capryloyl salicylic acid (Trade name: Mexoryl SAB); see page 139 of the International Cosmetic Ingredient Dictionary, 6th Edition, Volume 1, published by the Cosmetic Toiletries, and Fragrance Association, 1995, incorporated herein by reference.

With regard to formula I above, the R group contains from 2 to 22 carbon atoms, inclusive of each and every carbon atom in between this range, including subranges. Useful carbon numbers include 4, 6, 8, 10, 12, 14, 16, and 18. For the R′ group of formula I above, each and every carbon number between 1 and 18 is specifically included, and as are all subranges. Useful carbon numbers include 2, 4, 6, 8, 10, 12, 14, and 16. All odd carbon numbers between 2 and 22 carbon atoms for R, and all odd numbered carbon numbers between 1 and 18 for R′, are also specifically included.

Useful salts of the invention salicylic acid derivative may be obtained by salification with a base. Useful bases include inorganic basis such as alkali and alkaline metal hydroxides (sodium hydroxide, potassium hydroxide, and the like) or ammonia hydroxides. Organic bases may also be used for salification. Also useful are amphoteric bases. See U.S. patent application Ser. No. 08/627,965, incorporated herein by reference, for useful salicylic acid derivatives and useful salts thereof. Quatemium salts such as dimethylhydroxypropyl ammonium salts are also particularly useful.

In the present invention, the chemical skin peel compositions comprise the at least one salicylic acid derivative of the invention in a preferably dermatologically acceptable carrier, preferably in large amounts to provide a highly concentrated solution of salicylic acid derivative(s). The total concentration of salicylic acid derivative in the dermatologically acceptable carrier in this invention is preferably at least 0.5 wt %, more preferably at least 15 wt %, most preferably at least 20 wt % salicylic acid derivative, based on total weight of the composition. More preferably, the salicylic acid derivative(s) concentration in the carrier is at least about 25 wt % and most preferably is at least about 30 wt %, based on total weight of the composition. Generally, concentrations of over 35-36% are not preferred. (All weight percentages in this specification referring to the concentration of salicylic acid derivative or other components in solution are based on the total weight of the composition.) The upper limit of the concentration of salicylic acid derivative(s) according to the invention in the carrier is ordinarily limited to the saturation concentration in the carrier. The saturation concentration will ordinarily vary with temperature, generally being higher as the solution temperature is increased. The upper limit of invention salicylic acid derivative in the carrier is preferably limited to 35-36 wt % salicylic acid derivative, for those solvents in which the saturation concentration of the invention salicylic acid derivatives is greater than 35 wt %.

A group of preferred carriers used for the chemical skin peel compositions of the invention comprising one or more of the above salicylic acid derivatives are dermatologically acceptable liquid solvents in which the salicylic acid derivatives are soluble at high concentrations. The term “dermatologically acceptable liquid solvents” is intended to mean those solvents which can safely be used on the skin in the topical treatment method of this invention, i.e., solvents which do not provoke a severe reaction and which are not toxic when contacted with the skin for relatively short periods of time. Preferred solvents are organic solvents that are relatively volatile, to facilitate evaporation of the solvent after application of a coating of the salicylic acid derivative-containing solution onto the skin. Volatile solvents with moderate flash points, e.g., above 30° C., are preferred for safety reasons, to minimize flammability risks. Examples of preferred solvents include alkylene glycols such as propylene glycol, polyethylene glycol, and aqueous alcohol. Highly preferred solvents include ethanol and isopropanol. Other useful solvents include methanol, acetone and ether (diethyl ether). Mixtures containing one or more of these solvents or other solvents are included.

Ethanol is a highly preferred solvent. The ethanol may be aqueous ethanol, preferably containing about 85 to 99 wt % ethanol and more preferably containing about 90 to 95 wt % ethanol. The ethanol employed as the solvent is preferably a grade of ethanol suitable for use in dermatological formulations.

As indicated above for ethanol, carriers and solvents may contain water, which is preferably miscible with the solvent. The aqueous fraction of the solvent mixture is preferably minimized since its presence typically reduces the saturation concentration of salicylic acid derivative in the carrier/solvent, as the proportion of water is increased. The concentration of water is preferably not more than about 15 wt %, and more preferably not more than about 10 wt %, and most preferably not more than about 5 wt %.

It is important to note that the above-mentioned dermatologically acceptable liquid solvents, whether preferred or not, may be utilized alone or in combination with one another.

Other useful carriers herein include the various dermatological and cosmetic carriers such as gels, emulsions, creams, waxes, compacts, etc.

The chemical skin peel compositions of the present invention may be prepared by physically combining, blending, contacting, etc., in any order, one or more invention salicylic acid derivatives with the carrier, preferably the dermatologically acceptable liquid solvent, termed herein “mixing”. The salicylic acid derivative used may be in any form. However, the preferred form is a solid such as in crystalline or powdered form. The salicylic acid derivative and carrier/solvent may be provided in such relative amounts that provide the desired concentration. Alternatively, an excess of salicylic acid derivative may be provided and mixed with the carrier/solvent so as to provide a saturated salicylic acid derivative solution. Dissolution of the salicylic acid derivative may be promoted by mild heating of the carrier/solvent, with proper precautions being taken with those materials that have a very low flash point, i.e., solvents that are highly flammable.

The compositions of the invention may of course comprise other components, such as preservatives, stabilizers, antioxidants, thickening agents, surfactants, pigments, colorants, fragrances and other adjuvants. Such components are preferably dermatologically acceptable. Preferably, the additional components do not interfere with the efficacy or impose any negative influence upon the efficacy of the chemical peeling agent. Such additives may further include, for example, an aromatic, a surfactant, a preservative, an anti-oxidant, a moisturizing agent, and so on. In addition, vitamin A acid, an alkyl acrylatemethacrylate copolymer, etc. may be added. Of course the additional components may be present individually or in combination, and their concentrations are not limited and may be for example from about 0.01 to about 5 wt %, based on the weight of the chemical skin peel composition. In one embodiment the amounts of such additional components are minimized so as not to cause a significant reduction in the maximum, i.e., saturation, concentration of salicylic acid derivative in the solvent.

Other useful additives herein include compounds having the formula (II)

B[(CH₂CH₂O)_{m(AO)n—H]a}

where B is an alcohol residue,

AO is an alkylene-oxy group having from 3 to 18 carbon atoms

a in an integer that is greater than or equal to 1,

m is an integer that is greater than or equal to 4,

n is an integer that is greater than or equal to 0,

provided that a molar amount m of the ethylene oxide to be added is a value that amounts to at least 40% or the entire molecular weight of the ethylene oxide chain moiety.

In the above general formula II, the alcohol to be represented by reference symbol B is intended to mean a monovalent alcohol including, for example, an alkyl alcohol such as methanol, ethanol, butanol, lauryl alcohol, myristyl alcohol, palmityl alcohol, stearyl alcohol, cetyl alcohol, etc., and an alkenyl alcohol such as linoleyl alcohol, palmitoyl alcohol, oleyl alcohol, etc., a divalent alcohol such as ethylene glycol, propylene glycol, etc., a trivalant alcohol such as glycerin, trimethylol propane, triethanol amine, etc., a tetravalent alcohol such as pentaerythritol, diglycerin, etc. There may also be used other polyvalent alcohols such as sorbitol, polyglycerin and so on.

The alkyleneoxy group having 3 to 18 carbon atoms, as referred to by reference symbol AO, may include, for example, propyleneoxy, butyleneoxy, tetrahydrofuran, olefinoxy, and so on. The alkyleneoxy groups having 3 and 4 carbon atoms, such as oxidopropylene, oxidobutylene and tetrahydrofuran, are preferred.

In the above general formula II, reference symbol “a” is an integer of 1 or more. When the alcohol is a monovalent alcohol, the reference symbol “a” is 1. When the alcohol is a divalent alcohol, the reference symbol “a” is 2. Likewise, when the alcohol is a trivalent alcohol, the reference symbol “a” is 3. Further, when the alcohol is a polyvalent alcohol, the reference symbol “a” is the integer corresponding to the valence of the alcohol used.

In the above general formula II, reference symbol “m” is intended to mean an average molar amount of ethylene oxide to be added. The number of a polymerization chain of the ethylene oxide has to be at least 4.

Reference symbol “n” is intended to mean an average molar amount of an oxidized alkylene to be added. The number of a polymerization chain of the oxidized alkylene is zero or 1 or more.

The manner of polymerization of the ethylene oxide and the alkylene oxide is random or block polymerization.

The molar amount m of the ethylene oxide to be added is set to amount to 40% or more of the entire molecular weight of the ethylene oxide chain. This setting is based on the fact that, if the molar amount m of the ethylene oxide to be added would be less than the above molar amount, the phenol compound such as salicylic acid derivative would become unlikely to be sustained in the polyethylene glycol compound.

Materials having formula II may be synthesized in a conventional manner, for example, by reacting the ethylene oxide and the alkylene oxide with the alkyl alcohol or the alkenyl alcohol under an inert gas such as nitrogen or the like in the presence of a basic catalyst such as sodium hydroxide, potassium hydroxide or the like or an acidic catalyst such as boron tetrafluoride, tin tetrachloride or the like.

Specific examples of useful materials having formula II are discussed in EP 1 214 925 A1, incorporated herein by reference.

The invention compositions may contain other chemical peeling agents in addition to those specified above. Such agents are, for example, glycolic acid, trichloroacetic acid, salicylic acid, phenol, resorcinol, etc.

The composition according to the present invention may be applied in any manner, for example, by pasting, spraying, wiping, dispensing, etc. (hereinafter “applying”) the invention salicylic acid derivative(s) themselves or the invention chemical skin peel composition on the skin to be peeled. This can typically be accomplished with, for example, a spray bottle, an absorbent cotton swab wetted with the concentrated solution, with a solution-wetted sable brush or by gentle wiping with a solution-wetted absorbent fibrous material such as a gauze square or nonwoven pad, but other solution application techniques that coat the skin with the solution, preferably in a uniform manner, are also feasible. The application serves as a peel, the degree of which depends upon the amount or concentration of acid compound and time of application, all of which is within the skill of the ordinary artisan in view of this disclosure. The compound(s)/composition of the invention may in addition be applied not only to the skin to be peeled but also to surrounding areas.

The applied compound or composition is normally allowed to air dry over a relatively short period of time, preferably being less than 15 minutes and, with the preferred ethanol solvent, typically being in the range of about 3 to 10 minutes. Drying may be promoted by directing a gentle stream of air, preferably warm air, onto the treated area or by other analogous procedures. A single uniform application of the composition to the skin to be treated and/or its surroundings is generally sufficient. Additional or multiple applications either before or immediately after the applied solution has dried are normally unnecessary but may be useful in some situations, e.g., in treating skin on other parts of the body other than the face or in treating skin severely in need of peeling.

Once the applied solution has been on the skin of, e.g., the face for sufficient time the compound(s)/composition can be removed from the skin, for example after the composition has dried on the skin, or it may be allowed to remain on the skin for further time, depending on the results desired. When treatment is finished the skin may thereafter be preferably wiped or washed, rinsed, etc., with water etc. to remove any residue or traces of the applied salicylic acid derivative, composition or solution, including any deposits of salicylic acid derivative that may remain after drying. This step, however, is not critical but is highly preferred.

In situations where a relatively nonvolatile solvent is employed and the concentrated solution does not quickly dry on the treated skin, the skin is preferably wiped or washed, rinsed, etc., no later than about one hour, and preferably no more than about 15 minutes, after application of the concentrated salicylic acid derivative solution, to remove all traces of the applied solution. This period, of no more than about one hour, and preferably no more than about 15 minutes, is normally more than sufficient to provide the desired benefits resulting from treatment according to this invention, but is not limiting. Time periods can vary widely, as noted above.

Preferably, the treated skin is washed or wiped with water, e.g., with a water-moistened or water-wet swab, gauze square, or the like. Other solutions, such as an aqueous solution of mild detergent, aqueous alcohol solutions or isopropanol or ethanol, and the like, may also be used for this purpose. Additional applications of the concentrated salicylic acid derivative or composition immediately after the wiping/washing step, followed by drying and repeated wiping/washing, are generally unnecessary, as noted above, but may be desirable in some circumstances.

During the period generally beginning a few days, e.g., about 2 to 5 days, after the invention treatment, a typical patient may experience some peeling and scaling of the treated skin. The peeling and scaling may generally last no more than about 7 days and may be as short as 2 or 3 days in duration. Although the present invention does not require any special steps to be taken during this period, a bland or mild moisturizer may be applied, as desired, to the treated skin to reduce the visibility of scaling, peeling skin and to reduce skin dryness. The skin treated in the method of this invention may be treated further, with conventional skin treatment therapies.

It is to be noted herein that it is possible to apply to the skin the invention compound(s)/composition after removal of the cuticle, particularly at low amounts/concentrations whereby the cuticle remaining in the hair follicle or in the skin can be removed.

The compound(s)/composition of the invention is preferably applied to the skin to be peeled at a temperature of about 15° C. to about 30° C., about 20° C. to about 25° C. being preferred. Depending on carrier/solvent volatility characteristics, a temperature outside of these ranges, e.g., use of lower temperatures for highly volatile materials, may be preferred.

The skin to be peeled according to this invention is preferably first cleaned for example with ethanol, but this step is optional and not essential to the method of this invention. The cleaning may be accomplished by gently wiping the skin with a gauze square wetted with ethanol or acetone for example, before application of the compound(s)/composition is begun. This cleaning is intended to degrease the skin and to remove makeup and debris, as well as sebum. Other cleaning or degreasing agents may also be used. Other conventional skin preparation techniques may also be used in advance of the skin treatment according to this invention.

While not bound by theory, it is believed that the invention method can remove the epidermis (mainly the cuticle) of the skin and impose influences upon the cells of the stratum spinosum epidermidis and the stratum basale epidermidis of the epidermis, and cause the reproduction of the fibroblast of the corium. The aged corium portion can be replaced with the reproduced fibroblast. At the same time, the cuticle of the hair follicle can also be peeled off and the accumulated cuticle can be removed. The benefits of chemical peels are further discussed in WO 97/28786, incorporated herein by reference.

The compounds and compositions of the invention provide an advantage in that the treatment time, i.e., the period during which the treated skin is exposed to the salicylic acid derivative optionally in the carrier, is normally self-limiting and is not dependent on the intervention of the applicator for determining length of treatment time or determining when the treatment period should terminate. For relatively volatile solvents such as ethanol, the evaporation of the solvent from the coating of concentrated salicylic acid derivative solution is effective for controlling the treatment time, ensuring not only constancy in treatment time, but also avoiding the need for applicator intervention to avoid excessively long exposure to the solution of concentrated salicylic acid derivative.

A related benefit is the ease of ensuring that a uniform application of concentrated salicylic acid derivative is made on the area of skin to be treated. When the volatile solvent evaporates, the treated skin presents the appearance of having a white frosting from the residual salicylic acid derivative. Areas of skin to be treated which have been missed during application of the concentrated salicylic acid derivative are easily discerned, and inadvertent second applications of the concentrated salicylic acid derivative solution can also be readily avoided.

The treatment time according to this invention is preferably measured as the time of exposure of the treated skin to the compound/composition, with treatment time ending for compositions once the carrier (e.g., volatile solvent) has evaporated from the applied solution or once the still-wet coating of applied composition is wiped or otherwise removed from the skin.

The present invention is explained in more detail with the aid of the following embodiment examples. As used herein “Lipophilic Hydroxy Acid” and “LHA” refer to 5-n-octanoyl salicylic acid (capryloyl salicylic acid). Percents are weight percents based on total weight unless otherwise specified.

### EXAMPLES

### Example 1

36% Lipophilic Hydroxy Acid is mixed with ethanol.

### Example 2

35% Lipophilic Hydroxy Acid is mixed with a blend of ethanol/propylene glycol.

### Example 3

0.5% Lipophilic Hydroxy Acid is mixed with 20% glycolic acid in Ethanol 33%/PEG 300 30%/Glycerin 5%/Water QSP 100%

### Example 4

1.0% Lipophilic Hydroxy Acid is mixed with 20% glycolic acid in Ethanol 34%/PEG 300 30%/Glycerin 5%/Water QSP 100%.

### Example 5

2.0% Lipophilic Hydroxy Acid is mixed with 20% glycolic acid in Ethanol 30%/PEG 300 25%/Water QSP 100%.

### Example 6

0.5% Lipophilic Hydroxy Acid is mixed with 30% glycolic acid in Ethanol 28%/Water QSP 100%.

### Example 7

1.0% Lipophilic Hydroxy Acid is mixed with 30% glycolic acid Ethanol 31%/PEG 300 25%/Water QSP 100%.

### Example 8

2.0% Lipophilic Hydroxy Acid is mixed with 30% glycolic acid in Ethanol 31.5%/PEG 300 25%/Water QSP 100%.

### Example 9

0.5% Lipophilic Hydroxy Acid is mixed with 20% salicylic acid in Ethanol QSP 100%.

### Example 10

1.0% Lipophilic Hydroxy Acid is mixed with 20% salicylic acid in Ethanol QS 100%.

### Example 11

2.0% Lipophilic Hydroxy Acid is mixed with 20% salicylic acid in Ethanol QS 100%.

### Example 12

0.5% Lipophilic Hydroxy Acid is mixed with 30% salicylic acid in Ethanol QS 100%.

### Example 13

1.0% Lipophilic Hydroxy Acid is mixed with 30% salicylic acid in Ethanol Qs 100%.

### Example 14

2.0% Lipophilic Hydroxy Acid is contacted with 30% salicylic acid Ethanol Qs 100%.

### Example 15

10% Lipophilic Hydroxy Acid is mixed with alcoholic media.

### Example 16

2.0% Lipophilic Hydroxy Acid is mixed with glycolic media.

### Example 17

LHA at 10% in alcoholic or glycolic media is applied on skin:

Lipophilic Hydroxy Acid 10% in alcohol

The volar forearms of 4 people were cleansed using alcohol on gauze. The solution was then applied to a 4×4 cm area on the lower volar forearms. The solution remained on the forearms for five minutes and then was rinsed with water and NuGauze.

2.0% Lipophilic Hydroxy Acid is mixed with 30% glycolic acid in Ethanol 31.5%/PEG 300 25%/Water QSP 100%

The volar forearms of 4 people are cleansed using alcohol on gauze. The solution is applied to a 4×4 cm area on the lower volar forearms. The solution remains on the forearms for five minutes and is rinsed with water and NuGauze.

### Example 18

LHA at 5% in carrier is applied on skin for 4 minutes.

This specification fully describes the present invention and enables one of ordinary skill in the art to make and use the invention as set forth above, including a method of peeling skin comprising applying to skin to be peeled at least one salicylic acid derivative of formula (I):

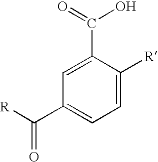

wherein

R is a linear, branched or cyclic saturated aliphatic group or an aliphatic unsaturated group containing one or a number of double bonds, which may or may not be conjugated, these groups containing from 2 to 22 carbon atoms and being able to be substituted by at least one substituent selected from the group consisting of (a) halogen atoms, (b) a trifluoromethyl group, (c) hydroxyl groups in the free form or esterified by an acid having from 1 to 6 carbon atoms, and (d) a carboxyl functional group which is free or esterified by a lower alcohol having from 1 to 6 carbon atoms;

R′ is a hydroxyl group or an ester functional group of formula

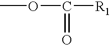

wherein R₁ is a linear or branched saturated or unsaturated aliphatic group having from 1 to 18 carbon atoms, and salts thereof;

in an amount sufficient, and for a time sufficient, to provide a superficial, medium or deep chemical peel. This method may be accomplished wherein said salicylic acid derivative is present in a composition comprising at least 1%, 2%, etc. by weight of said salicylic acid derivative based on total weight and a dermatologically acceptable carrier, and using, e.g., 5-n-octanoyl-salicylic acid. The composition may further comprise any of, e.g.,:

(i) a compound of formula (II)

B[(CH₂CH₂O)_{m(AO)n—H]a}

wherein B is an alcohol residue,

AO is an alkylene-oxy group having from 3 to 18 carbon atoms

a in an integer that is greater than or equal to 1,

m is an integer that is greater than or equal to 4,

n is an integer that is greater than or equal to 0,

provided that a molar amount m of the ethylene oxide to be added is a value that amounts to at least 40% or the entire molecular weight of the ethylene oxide chain moiety,

(ii) alkylene glycol,

(iii) glycolic acid, and

(iv) salicylic acid,

and/or polyethylene glycol, aqueous ethanol (e.g., 85% and greater aqueous ethanol), isopropanol, methanol, acetone, diethyl ether, and propylene glycol, salicylic acid, glycolic acid, etc.

1. A method of peeling skin, comprising applying to skin to be peeled at least one salicylic acid derivative of formula (I): 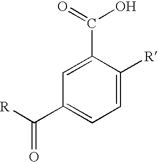
wherein
R is a linear, branched or cyclic saturated aliphatic group or an aliphatic unsaturated group containing one or a number of double bonds, which may or may not be conjugated, these groups containing from 2 to 22 carbon atoms and being able to be substituted by at least one substituent selected from the group consisting of (a) halogen atoms, (b) a trifluoromethyl group, (c) hydroxyl groups in the free form or esterified by an acid having from 1 to 6 carbon atoms, and (d) a carboxyl functional group which is free or esterified by a lower alcohol having from 1 to 6 carbon atoms;
R′ is a hydroxyl group or an ester functional group of formula 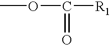
wherein R₁ is a linear or branched saturated or unsaturated aliphatic group having from 1 to 18 carbon atoms, and salts thereof;
in an amount sufficient, and for a time sufficient, to provide a superficial, medium or deep chemical peel.

2. The method according to claim 1, wherein said salicylic acid derivative is present in a composition comprising at least 1% by weight of said salicylic acid derivative based on total weight and a dermatologically acceptable carrier.

3. The method according to claim 2, wherein the salicylic acid derivative is present at an amount of at least 2% by weight.

4. The method according to claim 1, wherein the salicylic acid derivative is 5-n-octanoyl-salicylic acid.

5. The method according to claim 2, wherein the composition further comprises a compound selected from the group consisting of:
(i) a compound of formula (II)
B[(CH₂CH₂O)_{m(AO)n—H]a}
wherein B is an alcohol residue,
AO is an alkylene-oxy group having from 3 to 18 carbon atoms
a in an integer that is greater than or equal to 1,
m is an integer that is greater than or equal to 4,
n is an integer that is greater than or equal to 0,
provided that a molar amount m of the ethylene oxide to be added is a value that amounts to at least 40% or the entire molecular weight of the ethylene oxide chain moiety,
(ii) alkylene glycol,
(iii) glycolic acid, and
(iv) salicylic acid.

6. The method according to claim 2, wherein the composition comprises polyethylene glycol.

7. The method according to claim 2, wherein the composition comprises aqueous ethanol.

8. The method according to claim 7, wherein the aqueous ethanol comprises at least 85% ethanol.

9. The method according to claim 2, wherein the dermatologically acceptable carrier is selected from the group consisting of ethanol, isopropanol, methanol, acetone, diethyl ether, and propylene glycol.

10. The method according to claim 2, wherein the composition further comprises salicylic acid.

11. The method according to claim 2, wherein the composition further comprises glycolic acid.
